(19) United States
(12) Patent Application Publication
(10) Pub. No.: US 2010/0068245 A1
(43) Pub. Date: Mar. 18, 2010

(54) PREPARATION AND STORAGE OF STABLE, BIOLOGICALLY ACTIVE MATERIALS

(76) Inventor: Christian D. Manders, Pittsburgh, PA (US)

Correspondence Address: THE SONI LAW FIRM, 35 North Lake Ave., Suite 720, PASADENA, CA, 91101, US

(21) Appl. No.: 12/293,080

(22) PCT Filed: Mar. 15, 2007
(86) PCT No.: PCT/US2007/006592

§ 371 Date: Feb. 26, 2009

Related U.S. Application Data
(60) Provisional application No. 60/782,420, filed on Mar. 15, 2006.

(51) Int. Cl. A61K 9/70; B05D 3/06; C12N 5/071; A61K 38/43; A61K 45/00; A61K 38/00; A61P 17/00

(52) U.S. Cl. 424/443; 427/2.31; 435/325; 424/94.1; 424/93.1; 514/2

(57) ABSTRACT

A method for the preparation of biologically active materials is presented. The invention involves taking a base material such as allografts, xenografts, polymers, metals, and ceramics and combining it with a biologically active agent, such as proteins, cytokines, growth factors, and enzymes after which it is irradiated with ionizing radiation to sterilize and stabilize the material. The resulting biologically active material may then be stored at ambient temperature while maintaining its biological activity and the structural integrity of the base material. The invention is particularly useful for eliciting desired biological responses in human and animal medicine, and in certain industrial applications.

### CROSS-REFERENCES TO RELATED APPLICATION

This application claims the benefit of U.S. Provisional Application Ser. No. 60/782,420; filed Mar. 15, 2006, the content of which is hereby incorporated herein by reference.

### BACKGROUND OF THE INVENTION

The present invention relates to a method to prepare biologically active materials formed from the combination of a base material(s) and a biologically active agent(s). The base material may be comprised of such elements as human allografts, xenografts derived from mammals, reptiles, birds, amphibians, fish, and invertebrates, both naturally occurring and synthetic polymeric materials, metals, and ceramics. The biologically active agents include such elements as proteins, growth factors, cytokines, compounds, and/or drugs. This invention describes the preparation of the base material, such as a human allograft, xenograft, natural and synthetic polymeric materials, metals, and ceramics with the addition of biologically active agents, including but not limited to proteins, growth factors, cytokines, compounds, and/or drugs bound to the base material that is then irradiated with ionizing radiation so as to sterilize and stabilize the combined material. These combined materials are able to be stored at ambient temperature and to elicit biological responses in the person or animal, or industrial process into which or onto which the combined material is placed. The present invention is further directed at creating a biologically active material configured with biologically active agents such as proteins, growth factors and/or cytokines bound to a base material such that the biologically active agent adheres to, coats, or is embedded within the base material. The contents of U.S. Pat. Nos. 5,534,026 and 5,697,383 and U.S. Publ. No. 2005/0043,235 are each hereby incorporated herein by reference for all purposes.

By way of background, allograft skin has been shown to provide an excellent temporary skin coverage for burn patients, acting as a biological dressing. Allograft skin protects the wound from desiccation, contamination, and decreases wound pain. When allograft skin shows general adherence to a burn wound and evidence of graft vascularization within 48 to 72 hours of application, one can anticipate an excellent take of autograft skin applied to the wound following removal of the allograft skin. Limitations of fresh allograft skin includes the dearth of material, the need for refrigerated storage facilities, and a limited “effective” shelf life of approximately seven to ten days when the tissue is stored at 4 degrees Celsius. The possibility of disease transmission requires careful donor selection [Pruitt, B A et al., Arch. Surg. 119, 312 322, (1984)]. Other allograft materials such as bone and soft tissues face similar storage limitations.

Current developments in the field of allograft skin products focus on culturing epidermal cells to form skin like coverings to be used as skin allografts as referenced in U.S. Pat. No. 5,015,584. Cryopreservation of allograft is commonly used, which retains the viability of the donor cells to some extent. It was previously believed that living cells were required for the success of skin allograft. However, good results have been obtained using methods which preserve the allograft without retaining the viability of the cells, such as preservation with glycerol [Kreis R W, et al., J. Trauma 29(1), 51 54 (1989)] [Hermans, M H E, Burns 15(1), 57 59 (1989)], silicone fluid [Ballantyne, D L Jr. et al., Cryobiology 8, 211 215, (1971)] or lyophilization [Young, J M et al., Arch. Surg. 80(Feb.), 208 213, (1960)].

Fresh frozen allograft skin and lyophilized allograft skin have limitations such as demanding processing procedures. The requirements for such procedures confine the preparation of either material to special centers having proper facilities. The lyophilized material has an essentially unlimited nonrefrigerated shelf life, while the frozen material has a similarly prolonged shelf life provided proper refrigeration is maintained. Either material can be easily and rapidly prepared for use by rehydration or thawing. Lyophilized allograft skin generally adheres less well to the wound and is less able to reduce the bacterial count on the wound surface than fresh allograft skin [Pruitt, B A et al., Arch. Surg. 119, 312 322, (1984)].

U.S. Pat. Nos. 3,645,849 and 3,743,480 describe processes for sterilization of biological material (e.g., blood serum) by microwave irradiation. Methods for preparing and sterilizing biological tissues such as heart valves, veins, cartilage, ligaments and organs for use as bioprostheses are described in U.S. Pat. No. 4,994,237. The source of irradiation is a microwave oven. This method tends to heat the specimen and destroy its structure. A method of sterilization of biological material by ultraviolet light is described in U.S. Pat. No. 4,880,512. Ultraviolet light is an efficient method of sterilization but it does not penetrate through objects such as skin very well. Consequently, this method is not always secure. In addition, Ultraviolet Light is not efficient for batch sterilization.

Another widely used method of biological tissue preservation and sterilization, which does not retain cell viability, is gamma irradiation. This method has been used extensively in the preservation of bone allograft, with good results. It has also been used in the preservation of donor cartilage [Dingman R O et al., Plast. Reconstr. Surg. 28(5), 562 567, (1961)], blood vessels, heart valves [Wright K A et al., Sterilization and Preservation of Biological Tissues by Ionizing Radiation. Vienna, International Atomic Energy Agency, 107 118, (1970)], dura mater, and sclera [Colvard D M et al., Am. J. Ophthal., 87(4), 494 496, (1979)]. Irradiation sterilization of the tissue permits storage at room temperature, a considerable advantage when low temperature storage is unavailable. U.S. Pat. No. 4,351,091 employs gamma and x ray irradiation to preserve a corpse to kill bacteria and other microorganisms that contribute to the decomposition of a corpse. This patent does not address infectious diseases such as viruses or the feasibility of preparing or preserving the corpse for organ donation.

With the use of allograft skin, there is an associated risk of the transmission of disease, including the human immunodeficiency virus (HIV). Skin banks around the world were virtually closed down for two or more years after the reported transmission of HIV from allograft skin [Clarke J A, Lancet 1,983, (1987)]. Gamma irradiation at ranges of 250,000 cGy to 2.5 million cGy has been shown to inactivate HIV [Hiemstra H et al., Transfusion, 31(1), 32 39, (1991)] [Spire B et al., Lancet, 1, 188 189, (1985)]. The effect of gamma irradiation on human coagulation factors found in human plasma and on virus suspended in plasma or other types of suspending medium has been studied [Kitchen, A D et al., Vox Sang 56, 223 229, (1989)].

The base materials including, allografts, xenografts, polymeric materials, metals, and ceramics often lack biologically active agents that may be lost due to processing or are not naturally occurring. The present invention enhances the base materials such as allografts, xenografts, polymeric materials, metals, and ceramics for implant or surface usage by adding biologically active agents to them. The addition of these biologically active agents can greatly increase the functionality of the combined material when used in or on the body or in some cases when used in industrial processes for catalysis, fermentation, and other reactions. The base material may be in the solid, liquid, or aerosol state.

What is needed and heretofore unavailable is the creation of a biologically active material that combines a base substrate material with the addition of biologically active agents which may not be naturally occurring on the base material or may not be present in the desired concentrations. This allows for creating custom-made biologically active materials to better achieve prescribed effects. These biologically active materials will also be stable and storable at ambient temperature for a sustained period of time.

### SUMMARY OF THE INVENTION

In accordance In accordance with the present invention, a new method for the preparation, stabilization, and sterilization of biologically active materials is presented. This invention describes the preparation of a human allograft (including but not limited to skin, bone, tendon, fascia, cartilage, nerves, vessels, valves, corneas, organs, and component tissues of organs), xenograft (including but not limited to skin, bone, tendon, fascia, cartilage, nerves, vessels, valves, corneas, organs, and component tissues of organs), a natural or synthetic polymer, metals, and/or ceramics that includes the addition of biologically active agents including but not limited to proteins, polypeptides, and/or peptides such as growth factors and/or cytokines bound to the material. This biologically active material when introduced into or onto the body can affect the body in a desired way (including, but not limited to accelerating, inhibiting, or maintaining in an unaltered state, healing, vascularization, fibrosis, cell proliferation, cell death, and/or an immunologic response). The addition of the peptide, polypeptide, or proteinaceous growth factors and/or cytokines or drug entities (henceforth “biologically active agents”) will be capable of eliciting a biological response and the combined material will be storable at ambient temperature following processing, and may be or may not be sterile. The present invention is a combination of these two elements, (a) biologically active agents or drugs and, (b) a base material formed from allograft, xenograft, polymeric materials, metals, and/or ceramics that will be stable at ambient temperature following irradiation and that will produce a biologically active material that would elicit a biological response in treating a person or animal or as an industrial tool. This is an improvement on the prior art, which does not allow for sustained storage and stability of materials that include biologically active agents, in particular proteins, peptides, or polypeptides at ambient temperature. In addition, the present invention provides for a sterile and stable allograft, xenograft, polymeric material, metal, and/or ceramic and the attachment of the biologically active element to the base material prior to or following irradiation.

The method and products of the present invention have applications in many areas. In the case of skin, such applications include, but are not limited to, wound and burn therapy, venous stasis ulcers, diabetic foot ulcers, full thickness ulcers, Mohs surgery sites, skin graft donor sites, partial thickness wounds, areas of dermabrasion, temporary coverage of exposed abdominal viscera including small bowel and liver, exposed pericranium and cranium, fasciotomy sites, as a “Canary Test” on a wound bed before autografting, and areas of excision which are not closed pending final pathology report. The allograft or xenograft skin may be coated combined with a biologically active element agent that induces cell proliferation and increases healing rates or one that reduces scarring. For instance, VEGF may be attached to or combined with allograft, or xenograft, polymeric materials, metals, or ceramics and irradiated to allow the combined material to be stable at ambient or room temperature. The VEGF could help cell proliferation to close the wound while the allograft, xenograft, or other material skin would provide an occlusive wound covering that would create the ideal wound healing environment and would prevent the wound from drying out.

In the case of musculoskeletal allografts or xenografts, such applications could include bone grafts including but not limited to osteochondral grafts and chondral grafts, tendon grafts, nerve grafts, cartilage grafts, etc. These grafts may be coated, embedded, or bound with a biologically active element that will create an action when used on a patient. Bone grafts could be implanted with additional bone morphogenic proteins to speed the induction of bone formation.

In the case of natural or synthetic polymeric materials, metals, and ceramics the material could be used as an implantable material or as a surface covering. The polymeric material could be constructed in various shapes, forms, and consistencies to create the desired material properties for each individual application. Polymers from biological sources that can be utilized include, but are not limited to: Polygalacturonic acid, Hydroxypropyl cellulose, Hydroxyethyl cellulose, Heparin, Collagen, Gelatin, Carboxymethyl cellulose, Pectin, Algin, Ethyl cellulose, Glycosaminoglycan, Chitin/Chitosan, and other polysaccharides. Suitable metals for use as a base material for the present invention include, but are not limited to, medical grade stainless steel, titanium, chrome vanadium steel, silver, platinum, gold, and nickel-titanium alloys, such as nitinol. Suitable ceramics for use as a base material for the present invention include, but are not limited to, alumina, zirconia, silicon nitride, silicon carbide, steatite and cordierite.

The biologically active material could also be used in industrial or manufacturing processes. The biologically active material could be an agent used to initiate chemical or biological processes or to catalyze materials. These biologically active materials could be used to better stabilize starch processing enzymes or proteases that are used in detergents. These materials could be altered to increase the temperature stability of the enzymes.

Ionizing radiation, such as Gamma Irradiation from a Cobalt 60 source, has been earlier shown to inactivate HIV and has been used previously to sterilize allografts of bone and other tissues, but has not previously been used to sterilize, stabilize, and preserve biologically active materials comprised of the combination of biologically active agents and base materials. Human allografts were irradiated in the present invention and applied as a temporary wound dressing on a skin graft donor site. When compared with a frozen skin allograft on the same recipient, the irradiated allograft proved to be as effective. It offers the potential of a low cost, safe and effective treatment that can be used widely and without extensive training or extensive facilities.

An object of this invention is to develop a method of sterilizing and storing a biologically active material so that the risk of transmission of infectious diseases, particularly bacterial, fungal, and viral diseases, is eliminated or significantly reduced. An additional object of this invention is to provide a method of preparing a biologically active material that is inexpensive and includes additional biologically active agents to enhance the base material's functionality in the patient and easily available to a large percentage of the medical community. Another object of this invention is to allow for the preservation of the biologically active materials without the need for refrigeration or other treatment which would result in additional expense.

Other features and advantages of the invention will become apparent from the following detailed description, which illustrates, by way of example, the features of the invention.

### DETAILED DESCRIPTION OF THE INVENTION

The present invention is directed to the use of ionizing irradiation (for example, gamma irradiation) to sterilize and prepare allografts from humans, xenografts, synthetic or naturally occurring polymers, metals, and ceramics that include the addition of biologically active agents such as proteins, peptides, polypeptides, or drugs for use as a biologically active material. Because of the risk of the transmission of infectious diseases such as HIV, hepatitis, and other bacterial, fungal, and viral diseases, the use of a safe, effective and inexpensive method of preparing a biologically active material has become apparent. There is also a need for shelf-stable biologically active materials to treat disease or for industrial applications. This invention describes the preparation of an irradiated material that includes biologically active agents such as proteins, growth factors, cytokines, and/or drug entities bound, attached, embedded to the material to elicit a biological response in the body. The addition of the biologically active agents will create a material that can elicit a specified response in the body.

Donor skin from an HIV and hepatitis negative donor was obtained from the skin removed during a thighplasty. This skin was harvested using a power dermatome and sheets of skin 0.014 in. thick were obtained. These were placed immediately in Tis-u-Sol (Baxter; Deerfield, Ill.), a balanced salt solution, and stored overnight at 4 degrees Celsius. The harvested skin was then rinsed three times in Tis-u-Sol, and divided into several groups. One sample was placed in a solution of Eagles Minimal Essential Medium and dimethyl sulfoxide (DMSO) and frozen in liquid nitrogen. One piece was placed directly in formalin, to serve as a control for histological studies. Other pieces were placed in Tis-u-Sol in glass or plastic containers for irradiation with 3.0 million cGy at 23 degrees Celsius using a Cobalt 60 source. The allograft skin may be placed in a wide variety of solutions including but not limited to: glycerol, balanced salt solutions, Wisconsin's solution, etc.

The present invention can be practiced by irradiating the material substrate and the added biologically active element for a period of time sufficient to provide a sterilizing and/or preserving dose of ionizing radiation, such as gamma radiation from a Cobalt 60 source. Accordingly, such dosage is calculated using ordinary and usual parameters (i.e., medium size, etc.) of dosimetry. Irradiation dosages, sufficient to effect sterilization, are known in the art. Other irradiation variables such as oxygen content, humidity, temperature, time, dose rate, can be altered so as to achieve the optimum dose. One of normal skill in the art will be capable of altering these variables so as to achieve a suitable result. Rinsing is not obligatory to practice the invention. As additional controls, several pieces of skin were left in Tis-u-Sol at 23 degrees Celsius both with and without antibiotics (5000 U/cc penicillin and 5000 mcg/cc streptomycin) for the amount of time required to irradiate the 3 million cGy samples. At the end of the irradiation period, a sample of the irradiated skin and a sample of each of the 23 degrees Celsius controls were cultured and placed in formalin for analysis. The remainder of the irradiated skin was stored at 23 degrees Celsius (room temperature) in the closed containers employed for the sterilization procedure and may be stored for an extended period of time.

It is contemplated by the present invention that the irradiated biologically active material made according to teachings of the present invention may be stored at ambient or room temperature for one day, two days, three days, five days, seven days, ten days, twenty days, thirty days, sixty days, one hundred eighty days, three hundred sixty five days, two years, and even longer. The storage time at ambient temperature will be dependent on the individual biologically active agents and the type of base material used. The finished biologically active material will be shelf-stable, storable at ambient temperatures and the biological activity will be stabilized such that the structural integrity of the material will be maintained with an enhanced biological activity after processing.

After 14 days, a sample of cryopreserved skin and two samples of the 3 million cGy irradiated skin were placed on a thigh skin graft donor site of a healthy volunteer. A portion of each allograft was placed in formalin for analysis at the time, and 2 mm punch biopsies were obtained at 3, 6, 8, 10, 13, 17, and 24 days post op. All samples were stained using hematoxylin and eosin, as well as colloidal iron, and all histological samples were numbered and evaluated in a blinded fashion.

Cultures were negative for bacteria for both the control samples and the irradiated samples.

Throughout the study, the patient reported minimal pain from all areas of his donor site; no evidence of infection was seen at any time.

The clinical course of the allografts showed that at postoperative day two, both grafts looked somewhat pink and were firmly adherent to the graft bed. At day three both grafts were still pink and intact, but some epidermolysis was visible on the frozen allograft. By postoperative day six, the superficial epidermis of the frozen allograft had almost completely sloughed, while in contrast the irradiated allograft remained intact and supple. Histological examination at this point shows the frozen allograft dermis overlying the patient's own epidermis and dermis, while the irradiated graft appears intact but with nonviable cells. Between postoperative day eight and thirteen, the frozen allograft began to develop some areas of epithelialization over the remaining allograft dermis, while the irradiated allograft began to form a thin eschar interspersed with some areas of epithelialization. By postoperative day seventeen the frozen allograft began to slough completely, while the site of the irradiated allograft was predominantly epithelialized, with some areas of eschar still remaining. Histologic examination shows the frozen allograft to be well epithelialized over the allograft dermis, with the patient's dermis and epidermis underneath; while the nonviable cells of the irradiated graft have been replaced with living cells. At postoperative day 27 the frozen allograft site still had many areas lacking epithelialization due to islands of retained allograft dermis, while the irradiated site was predominantly epithelialized.

We have shown that irradiated allograft is as effective a biological dressing as conventional frozen allograft. HIV and other viruses are inactivated by the radiation dose used in the present invention.

The results in this patient indicate that the cryopreserved allograft does indeed survive to form a viable skin layer over the patient's own tissue until it is rejected. The irradiated allograft forms an inert, protective barrier which sloughs after regrowth of the patient's own epidermis. Both forms of allograft performed well as a dressing, providing good coverage and pain relief as well as protection from infection. The irradiated allograft, however, produced a stable epithelial surface ten days before the cryopreserved allograft.

Skin allograft preservation by ionizing irradiation (for example, gamma irradiation) has many advantages, and makes skin allograft use a possibility in areas where it is not currently available, such as small hospitals, doctors' offices, and developing countries of the world. The preparation of irradiated skin allograft is inexpensive and simple to perform, requiring only basic materials and access to a source of ionizing radiation, such as Cobalt 60. Irradiated allograft can be stored on the shelf at room temperature and does not require liquid nitrogen or low temperature freezer storage. Application of irradiated skin requires no thawing, washing or rehydration, as found with other methods of skin preservation.

The only factors limiting the usefulness of this technique are the availability of cadaveric skin and a source of ionizing radiation, such as Cobalt 60. The low cost of the method and the fact that the skin is virus free, and specifically HIV free, will make this a most attractive method of preparing allograft skin for patients with burns and other wounds.

The present invention includes a method for the addition of biologically active agents such as proteins, growth factors, peptides, enzymes, and/or cytokines that favor wound vascularization and healing to a human skin allograft that can be irradiated (for example, terminal sterilization) and stored at room temperature. The method and product of the present invention combines these two elements, (a) a biologically active agent or agents such as proteins, growth factors, cytokines, enzymes, and drug entities and, (b) a base material such as allograft, xenograft, polymeric materials, metals, or ceramics both of which are room temperature stable after irradiation to provide a biologically active material to elicit a response in or on the body. The combination of a base material and biologically active agents provides a novel room temperature-stable preparation of a biologically active material. Heretofore, it was not understood that these entities could be combined, irradiated, stabilized, and stored at room temperature. Accordingly, it has been generally accepted that biologically active agents must be stored in the cold until used. The application of materials with biologically active agents incorporated into them provides a mechanism of delivering proteins and/or growth factors to wounds at biological temperatures. This invention therefore also provides the preparation and delivery mechanism of biologically active agents heretofore not available.

The methods and products of the present invention allow the simultaneous delivery of biologically active agents to wounds while providing an ideal closure for healing. The present invention could involve skin with the epidermal layer or only the dermal layer of the skin. This could prove an advantage for wounds that lack adequate vascularity or whose environment has diminished the supply of the usual factors present in a normally healing wound. The invention would uniquely provide an adherent wound closure and thereby an ideal healing environment, and at the same time it would also allow the ready delivery of growth factors that could accelerate or jump start wound healing in difficult wounds. As will be appreciated by those of ordinary skill in the art various methods, procedures and systems are available for providing a mechanism of addition and binding of the growth factors to the allograft.

The binding or attachment elements of the invention are subsequently described. The biologically active agents may be combined with the human allograft, xenograft, natural or synthetic polymeric material, metal, or ceramic by one or more, but not limited to, the following methods:

### COMBINATION OF BASE MATERIAL WITH BIOLOGICALLY ACTIVE AGENT

The combination of allograft and the biologically active agents such as proteins, growth factors, and cytokines may be made in several ways. Four such methods, which are not meant to be the only methods available, include simple adsorption and absorption, covalent bonding such as with formation of urethane bonds, and sequestration with formation of salts. Additionally, biologically active agents may be injected, inserted, or embedded into the base material.

### SIMPLE ADSORPTION OR ABSORPTION

The base material may be combined with biologically active agents by the act of simple immersion of the base material in a solution containing a suitable concentration of the biologically active agent(s) of interest. Such immersion may be conducted at temperatures from 0° to 40° C. for intervals of several seconds to hours and even days. The biologically active agents are bound by hydrogen bonding and ionic interactions and are therefore readily available for release in a therapeutic environment. The biologically active agents typically have charged groups like —N⁺H₃ and —CO₂—, and groups that are highly polar, such as —OH and —SH. Similar groups are found on allograft and xenograft materials and many natural and synthetic polymers, metals, and ceramics allowing binding interactions to occur with resultant immobilization of the desired biologically active agents on the base material of interest.

### COVALENT BONDING

Cytokines and growth factors commonly contain amine groups (—NH₂), sulfhydryl groups (—SH), carbonyl groups (—CO₂), and oxygen species (—O). Polyisocyanate species may react with acidic groups in the following way:

O═C═N-R-N═C═O+—XH

where X=—NH₂, —SH, —CO₂, —O. A preferred cross-linking agent is the polyether polyisocyanate sold as Hypol© Foamable Hydrophilic Prepolymer (W. R. Grace & Co., Lexington, MA). This produces a reaction:

RNCO+H₂O→RNHCOOH

(Unstable carbamic acid)

RNHCOOH→RNH₂+CO₂↑

(Amine formation and gas generation)

RNH2+RNCO→RNHCONHR

(Urea chain extension Cross-linking formation)

Other cross-linking agents may be suitable such as alkylene polyacrylates, alkylene polymethacrylates, alkylene glycolpolymethacrylates, polyaldehydes and other cross-linking reagents that will cross-link molecules with reactive protic groups. Suitable initiators of polymerization may be required, including as examples but not limited to azobisisobutylnitrile, peroxide initiators such as benzoyl peroxide, isopropyl peroxide and similar reagents. Such cross-linking will result in a covalent bond between the allograft, xenograft or polymeric material and the chosen growth factor or cytokine.

### SALT FORMATION

Growth factors and cytokines may be precipitated and bound by alkali metal phosphates. Calcium phosphate as hydroxyapetite is an example of a polymer capable of binding molecules to surfaces. This agent is utilized to bind a drug preventing fibrosis to drug eluting stents.

The base material can be loaded with the desired biologically active agent(s), which is believed to occur by ionic binding involving ionic sites on the biopolymer, with the desired bioactive agent, which may be macromolecules such as growth factors, antispasmodic agents, or any other active biological bioactive agent, such as adrenergic agents such as ephedrine, desoxyephedrine, phenylephrine, epinephrine and the like, cholinergic agents such as physostigmine, neostigmine and the like, antispasmodic agents such as atropine, methantheline, papaverine and the like, tranquilizers and muscle relaxants such as fluphenazine, chlorpromazine, triflupromazine, mephenesin, meprobamate and the like, antidepressants like amitriptyline, nortriptyline, and the like, antihistamines such as diphenhydramine, dimenhydrinate, tripelennamine, perphenazine, chlorprophenazine, chlorprophenpyradimine and the like, hyptotensive agents such as rauwolfia, reserpine and the like, cardioactive agents such as bendroflumethiazide, flumethiazide, chlorothiazide, aminotrate, propranolol, nadolol, procainamide and the like, angiotensin converting enzyme inhibitors such as captopril and enalapril, bronchodialators such as theophylline, steroids such as testosterone, prednisolone, and the like, sedatives such as chloral hydrate, phenobarbital and other barbiturates, glutethimide, analgesics such as aspirin, acetaminophen, phenylbutazone, propoxyphene, methadone, meperidine and the like, etc. These substances are frequently employed either as the free compound or in a salt form, e.g., acid addition salts, basic salts like alkali metal salts, etc.

The following are examples, which are illustrative and not intended to be limiting, of biologically active agents, including , but not limited to proteins, human growth factors, cytokines, enzymes, and toxins that could conceivably be combined with a base material of the present invention for benefit:

(1) HUMAN CHEMOKINES: Human BCA-1/BLC; Human BRAK; Human Chemokine CC-2; Human CTACK; Human CXCL-16; Human ELC; Human ENA; Human Eotaxin; Human Exodus-2; Human Fractalkine; Human GCP-2; Human GRO; Human HCC-1; Human HCC-4; Human I-309; Human IP-10; Human I-TAC; Human LAG-1; Human LD78-beta; Human LEC/NCC-4; Human LL-37; Human Lymphotactin; Human MCP; Human MDC; Human MEC/CCL28; Human MIG; Human MIP; Human NAP-2; Human PARC; Human PF-4; Human RANTES; Human SDF; Human TARC; Human TECK.

(2) HUMAN CYTOKINES: Human Cardiotrophin-1; Human CLC/NNT-1/BSF-3; Human CLF-1/CLC Complex; Human Cytokine Mixtures; Human EMAP-II; Human gAcrp30; Human Interferons; Human Interleukin Receptor Agonists; Human Interleukins; Human TNF Ligand Family; Human Tumor Necrosis Factors; Recombinant Human Thioredoxin.

(3) HUMAN GROWTH FACTORS: Human Amphiregullin; Human Angiogenesis Proteins; Human Betacellulin; Human BMP; Human Colony Stimulating Factors; Human Connective Tissue Growth Factor; Human Cripto-1; Human Cryptic; Human ECGF; Human EGF; Human EG-VEGF; Human Erythropoietin; Human Fetuin; Human FGF; Human GDF-11; Human GDF-15; Human Growth Hormone Releasing Factor; Human HB-EGF; Human Heregulin; Human HGF; Human IGF; Human Inhibin; Human KGF; Human LCGF; Human LIF; Human Miscellaneous Growth Factors; Human MSP; Human Myostatin; Human Myostatin Propeptide; Human Nerve Growth Factor; Human Oncostatin M; Human PD-ECGF; Human PDGF; Human PIGF; Human SCF; Human SMDF; Human Stem Cell Growth Factor; Human Thrombopoietin; Human Transforming Growth Factor; Human VEGF.

(4) ENZYMES: Dehydrogenase, Luciferase, and DMSO reductase, Alcohol dehydrogenase (NAD), Alcohol dehydrogenase (NADP), Homoserine dehydrogenase, Aminopropanol oxidoreductase, Diacetyl reductase, Glycerol dehydrogenase, Propanediol phosphate dehydrogenase, glycerol-3-phosphate dehydrogenase (NAD), D-xylulose reductase, L-xylulose reductase, Lactate dehydrogenase, Malate dehydrogenase, Isocitrate dehydrogenase, HMG-CoA reductase, Glucose oxidase, L-gulonolactone oxidase, Xanthine oxidase, Glyceraldehyde 3-phosphate dehydrogenase, Acetaldehyde dehydrogenase, Pyruvate dehydrogenase, Biliverdin reductase, Protoporphyrinogen oxidase, 5-alpha reductase, Monoamine oxidase, Dihydrofolate reductase, Methylenetetrahydrofolate reductase, Sarcosine oxidase, Dihydrobenzophenanthridine oxidase, NADH dehydrogenase, Urate oxidase, Glutathione reductase, Thioredoxin reductase, Sulfite oxidase, Cytochrome c oxidase, Deiodinase, Coenzyme Q-cytochrome c reductase, Catechol oxidase, Laccase, Cytochrome c peroxidase, Catalase, Myeloperoxidase, Thyroid peroxidase, Glutathione peroxidase, 4-hydroxyphenylpyruvate dioxygenase, Renilla luciferase, Cypridina luciferase, Firefly luciferase, Watasenia luciferase, Oplophorus luciferase, Cytochrome P450 oxidase, Aromatase, CYP2D6, CYP2E1, CYP3A4, Cytochrome P450 oxidase, Nitric oxide dioxygenase, Nitric oxide synthase, Aromatase, CYP2D6, CYP2E1, CYP3A4, Phenylalanine hydroxylase, Tyrosinase, Superoxide dismutase, Ceruloplasmin, Nitrogenase, Glutathione S-transferase, Catechol-O-methyl transferase, DNA methyltransferase, Histone methyltransferase, ATCase, Ornithine transcarbamoylase, Aminolevulinic acid synthase, Choline acetyltransferase, Factor XIII, Gamma glutamyl transpeptidase, Transglutaminase, Hypoxanthine-guanine phosphoribosyltransferase, Thiaminase, Alanine transaminase, Aspartate transaminase, Kinase, Protein kinase, CAMP-dependent protein kinase, Hexokinase, Glucokinase (hexokinase IV), Phosphofructokinase, Thymidine kinase, P53, PFP (enzyme), Tyrosine kinase, Insulin receptor, Creatine kinase, Adenylate kinase, DNA polymerase, DNA polymerase I, DNA polymerase III holoenzyme, Galactose-1-phosphate uridylyltransferase, Polymerase, Primase, Reverse transcriptase, Transposase, Hydrolytic enzyme, Nuclease, Endonuclease, Exonuclease, Acid hydrolase, Phospholipase, Cholinesterase, Lipoprotein lipase, Ubiquitin carboxy-terminal hydrolase Li, Phosphatase, Alkaline phosphatase, Fructose bisphosphatase, Phospholipase, CGMP specific phosphodiesterase type 5, Phospholipase, Restriction enzyme, Deoxyribonuclease, RNase H, Ribonuclease, Amylase, Sucrase, Chitinase, Lysozyme, Maltase, Lactase, Beta-galactosidase, Hyaluronidase, Alanine aminopeptidase, Angiotensin-converting enzyme, Serine protease, Chymotrypsin, Trypsin, Thrombin, Factor X, Plasmin, Acrosin, Factor VII, Factor IX, Factor XI, Elastase, Factor XII, Tissue plasminogen activator, Protein C,

Separase, Pepsin, Rennet, Renin, Trypsinogen, Plasmepsin, Matrix metalloproteinase, Metalloendopeptidase, Urease, Beta-lactamase, Arginase, Adenosine deaminase, GTP cyclohydrolase I, Nitrilase, Helicase, DnaB helicase, RecQ helicase, ATPase, NaKATPase, ATP synthase, Kynureninase, Ornithine decarboxylase, Uridine monophosphate synthetase, Aromatic-L-amino-acid decarboxylase, RubisCO, Carbonic anhydrase, Tryptophan synthase, Adenylate cyclase, Guanylate cyclase, Enoyl CoA isomerase, Protein disulfide isomerase, Phosphoglucomutase, Topoisomerase (type I:, type II:), Argininosuccinate synthetase, CTP synthase, Pyruvate carboxylase, Acetyl-CoA carboxylase, DNA ligase.

(5) PROTEINS: Fibrous proteins such as: tubulin, actin, keratin, myosin, Tau, dystrophin; Extracellular matrix proteins such as: collagen, elastin, reelin; Plasma proteins such as: albumin, serum amyloid P component, fibrin, thrombin, Factor XIII, protein C, protein Z, protein Z-related protease inhibitor, protein S, complement proteins, c-reactive proteins, hemoglobin, myoglobin, cadherin, integrin, NCAM, selectin, Transmembrane transport proteins such as: CFTR, glycophorin C, scramblase, Acetylcholine receptor, potassium channel, glucose transporter; Hormones and growth factors such as: oxytocin, insulin, epidermal growth factor, insulin-like growth factor; Receptor proteins such as G-protein-coupled receptor, estrogen receptor, histones, Cprotein, C-myc, MyoD, FOXP2, FOXP3, P53; Immune system proteins such as: immunoglobins, T cell receptor, major histocompatibility antigens, ferritin.

(6) TOXINS: neurotoxins such as species of Botulinum toxin; biotoxins; hemotoxins; necrotoxins.

### FIRST EXAMPLE

### Allograft Skin

Allograft skin may be combined with VEGF (Vascular Endothelium Growth Factor) and then packaged and irradiated with production of a sterile allograft storable at ambient temperature and possessing an enhanced ability to nourish the growth of new vessels in a wound to which it is applied. This is accomplished by rinsing recovered allograft skin to wash off any antibiotics and freezing medium that may be present. One then places the allograft dermis-side down on a piece of Telfa pad saturated with a solution of VEGF at a concentration of 5 to 70 nanograms per ml in a balanced salt solution or other liquid media. The skin is allowed to absorb the VEGF solution for 15 minutes at room temperature. The skin is then packaged in a moist dressing and sealed in a packaged made of a composite of plastic and foil. This is sealed and them irradiated with at least 30 kGy of ionizing radiation. After this last step, the skin can be stored at ambient temperature.

### SECOND EXAMPLE

### Allograft Bone

Allograft bone is commonly used to aid in the reconstruction of fractures and in the successful fusion of a patient's bone, The growth of vessels into the area of healing can significantly assist in the rapid union of the graft and the native bone being treated. Increased vascularity brings protective white cells and antibiotics and encourages a vigorous metabolism that accelerates the bony healing and consolidation of graft and recipient site.

For this embodiment small pieces of allograft bone from 1 to 5 mm in diameter are simply immersed in a solution of VEGF with a concentration of 5 to 70 nanograms per ml in a balanced salt solution. The fragments are then lifted from the solution and allowed to drain until moist but no longer dripping. The treated bone allograft is then placed in a suitable container and sealed in an impervious container which may be a bottle or a bag. The container is then subjected to 30 kGy of ionizing radiation after which the allograft and the adsorbed VEGF are stable at room temperature for an extended period of time.

### THIRD EXAMPLE

### Pollulan Polymer as VEGF Carrier

Pollulan is a biological biodegradable polymer that may be formed into a wafer which can serve as a delivery vehicle. In this application a wafer of the polymer of size chosen is immersed in a solution of VEGF with a concentration of 5 to 70 nanograms per ml for 15 minutes at room temperature. The wafer is then lifted from the bath and allowed to drain and then covered with a plastic sheet which is then placed in a sealable container. The polymer carrier and its VEGF cargo are then irradiated with at least 30 kGy of ionizing radiation. Thereafter the package can be stored for extended periods of time at ambient temperature.

### RADIATION

Ionizing radiation may be administered by a source such as a commercial Cobalt 60 or electron beam source. The dose may be selected according to the needs of the material at hand. Bacterial sterilization may be accomplished with reference to tables of radiation sensitivity of bacteria and the need to reduce the bacterial count to less than 10-6 colony forming units. The bioburden present at the start is important for this calculation as is familiar to anyone skilled in the art of radiation sterilization. Biological samples may be sterilized of viruses if an adequate dose of radiation is selected. The common pathogens screened for in donor selection are eliminated by a cumulative dose of 30 kGy or more. Thus, high dose ionizing radiation is capable of sterilizing biological specimens and thereby may eliminate the risk of inadvertent infection by transplantation of allograft and xenograft materials. Appropriate doses may vary according to the needs of a particular situation, varying from 2000 cGy to over 50 kGy, with the most frequent dose being between 3 and 35 kGy.

Radiation may be administered at temperatures from the very cold (liquid nitrogen and dry ice) to room temperature and above. Rates of radiation delivery may vary from about 0.5 kGy/hr to about 4.0 kGy/min for a period of about 5 minutes to about 40 hours. Low temperature renders radiation less effective in inactivating bacteria and viruses. Someone skilled in the art of radiation sterilization knows how to adjust the dose administered to account for the potentially protective effects of low temperature.

Biological materials subjected to high dose irradiation may be stored at room temperature. The storage temperature includes temperatures from 0° to 40° C. The duration of storage may vary from 5 minutes, to 15 minutes, to 1 hour, to 12 hours, to 1 day, to 7 days, to 30 days, to six months, to 1 year, to 2 years, to 6 years and beyond, and intermediate times in between.

### METHODS OF USE

### Surface Application

Both acute and chronic wounds may benefit from growth factors and cytokines delivered in pharmacologic doses. As an example of this allograft skin delivering platelet derived growth factor (PDGF) would promote healing in chronic wounds as has been demonstrated for PDGF alone. Allograft would offer the additional advantages of closing the wound to bacteria invasion and preventing desiccation.

### IMPLANTATION

Musculoskeletal tissues are typically implanted in the body in an attempt to reconstruct or repair damaged elements of the musculoskeletal system. An example that could enjoy widespread use is bone allograft with bone morphogenetic protein (BMP). Such allograft would be incorporated much faster and would heal a fracture or fusion site much more securely.

### INDUSTRIAL USE

Industry makes widespread use of enzymes and fermentation. Fermentation in particular could be aided by the addition of a natural polymer such as a polysaccharide with embedded enzyme that would help hydrolyze the polysaccharide to present its constituent sugars as a substrate for fermentation. Prepared as described in this disclosure such a functional substrate would be sterile so the fermentation would not contaminated by unwanted bacterial growth.

### VETERINARY USE

Large animal veterinarians often must treat their animal patients with many of the technologies that are available to human patients. A fracture in a race horse's leg could be addressed with allograft bone enhanced with BMP. This would favor recovery and the preservation of a potentially very valuable animal for breeding, personal companionship, and possibly even resumption of racing.

While particular forms of the invention have been illustrated and described, it will also be apparent to those skilled in the art that various modifications can be made without departing from the inventive concept. References to use of the invention with a specific compound, chemical or radiation source and with respect to a particular disease or condition are by way of example only, and the described embodiments are to be considered in all respects only as illustrative and not restrictive. The present invention may be embodied in other specific forms without departing from its spirit or essential characteristics. Accordingly, it is not intended that the invention be limited except by the appended claims.

We claim:

1. A method of preparing a biologically active material, comprising the steps of:
providing a base material;
providing a biologically active agent;
combining the biologically active agent with the base material so as to form a biologically active material; and
exposing the biologically active material to a source of ionizing radiation sufficient to sterilize and stabilize the biologically active material.

2. The method of claim 1, wherein combining the biologically active agent to the base material includes using an adsorption process.

3. The method of claim 1, wherein combining the biologically active agent to the base material includes using an absorption process.

4. The method of claim 1, wherein combining the biologically active agent to the base material includes a using a covalent bonding process.

5. The method of claim 1, wherein combining the biologically active agent to the base material includes sequestration with salt formation.

6. The method of claim 1, further including storing the biologically active material at a temperature above freezing without substantial degradation of the base material or the biologically active agent, while maintaining sterility and stability of the biologically active material.

7. The method of claim 6, wherein storing the biologically active material is performed at ambient temperature for a period of at least one day.

8. The method of claim 1, wherein providing a base material includes using an allograft.

9. The method of claim 8, wherein using an allograft includes providing a material selected from the group consisting of skin, bone, tendon, fascia, cartilage, nerves, vessels, valves, corneas, organs, and component tissues of organs.

10. The method of claim 1, wherein providing a base material includes using a xenograft.

11. The method of claim 10, wherein using a xenograft includes providing a material selected from the group consisting of skin, bone, tendon, fascia, cartilage, nerves, vessels, valves, corneas, organs, and component tissues of organs.

12. The method of claim 1, wherein providing a base material includes using a polymer.

13. The method of claim 12, wherein using a polymer includes providing a material selected from the group consisting of Polygalacturonic acid, Hydroxypropyl cellulose, Hydroxyethyl cellulose, Heparin, Collagen, Gelatin, Carboxymethyl cellulose, Pectin, Algin, Ethyl cellulose, Glycosaminoglycan, Chitin/Chitosan, and polysaccharides.

14. The method of claim 1, wherein providing a base material includes using a metal.

15. The method of claim 14, wherein using a metal includes providing a material selected from the group consisting of medical grade stainless steel, titanium, chrome vanadium steel, silver, platinum, gold, and nickel-titanium alloys, such as nitinol.

16. The method of claim 1, wherein providing a base material includes using a ceramic.

17. The method of claim 16, wherein using a ceramic includes providing a material selected from the group consisting of alumina, zirconia, silicon nitride, silicon carbide, steatite and cordierite.

18. The method of claim 1, wherein providing a biologically active agent includes using a proteinaceous material.

19. The method of claim 1, wherein providing a biologically active agent includes using a material selected from the group consisting of a protein, a polypeptide, and a peptide.

20. The method of claim 1, wherein using a biologically active material includes providing a material selected from the group consisting of growth factors, cytokines, chemokines, enzymes, antispasmodic agents, tranquilizers and muscle relaxants, adrenergic agents, cholinergic agents, antidepressants, antihistamines, hyptotensive agents, cardioactive agents, angiotensin converting enzyme inhibitors, bronchodialators, steroids, sedatives, analgesics, proteins and toxins.

21. The method of claim 20, wherein using growth factors includes providing a material selected from the group consisting of Human Amphiregulin; Human Angiogenesis Proteins; Human Betacellulin; Human BMP; Human Colony Stimulating Factors; Human Connective Tissue Growth Factor; Human Cripto-1; Human Cryptic; Human ECGF; Human EGF; Human EG-VEGF; Human Erythropoietin; Human Fetuin; Human FGF; Human GDF-11; Human GDF-15; Human Growth Hormone Releasing Factor; Human HB-EGF; Human Heregulin; Human HGF; Human IGF; Human Inhibin; Human KGF; Human LCGF; Human LIF; Human Miscellaneous Growth Factors; Human MSP; Human Myostatin; Human Myostatin Propeptidc; Human Nerve Growth Factor; Human Oncostatin M; Human PD-ECGF; Human PDGF; Human PIGF; Human SCF; Human SMDF; Human Stem Cell Growth Factor; Human Thrombopoietin; Human Transforming Growth Factor; and Human VEGF.

22.-39. (canceled)

40. A biologically active material prepared according to claim 1.

41. (canceled)

42. A biologically active material, comprising:
a base material; and
a biologically active agent,
wherein the biologically active agent has been combined with the base material so as to form a biologically active material, and
wherein the biologically active material has been exposed to a source of ionizing radiation sufficient to sterilize and stabilize the biologically active material.
